(19) United States
(12) Patent Application Publication
(10) Pub. No.: US 2021/0052430 A1
(43) Pub. Date: Feb. 25, 2021

(54) APPARATUS FOR HANDS FREE APPLICATION OF COMPRESSION TO A WOUND SITE

(71) Applicant: Paula Riley, Pound Ridge, NY (US)

(72) Inventor: Paula Riley, Pound Ridge, NY (US)

(21) Appl. No.: 16/998,101

(22) Filed: Aug. 20, 2020

Related U.S. Application Data
(63) Continuation of application No. 62/890,374, filed on Aug. 22, 2019.

(51) Int. Cl. A61F 13/00; A61F 7/02

(52) U.S. Cl. CPC A61F 13/00; A61F 7/02; A61B 17/1327; A61F 2013/00187; A61F 2013/0028

(57) ABSTRACT

An adjustable hands-free compression device for application of a treatment to a locus of a wound. The compression device is capable of exerting compression on specific locations post-surgery after an injury or any trauma that requires compression to a specific point or area of the body to secure placement of therapeutic media such as an ice pack or post-surgical site or injury. The compression device has a body engagement band for bilateral adjustment engagement of a compression assembly track having independent adjustable compression engagement elements against the treatment device. As such, the structure will conform to the area of the body to which it is secured and allow for easy adjustment of the compression of the targeted location of the site and the amount of compression required to achieve the best outcome.

This application claims the benefit of U.S. Provisional Application No. 62/890,374, filed on Aug. 22, 2019.

### BACKGROUND OF THE INVENTION

### 1. Technical Field

The present invention relates to wound treatment and more particularly to apparatus for applying compression to a wound site.

For many injuries, a preferred treatment modality is the application of pressure to a wound site to staunch bleeding, reduce swelling and reduce contusions associated with a wound or injury. For example, soft and hard tissue biopsies and surgeries or injuries can cause swelling, contusion and bleeding requiring compression.

### 2. Description of Prior Art

Current compression products such as traditional adhesive bandages, elastic bandage wraps, silicone tape, or the like exert and evenly distribute compression over the entire contact area of a treatment device applied to the wound such as a bandage, an ice pack, heat pack or an electronic treatment device. Accordingly, these compression products are unable to apply controlled compression at specific points of post-surgical incision, injury or wound.

Devices with diffuse compression do not reduce swelling, contusion and bleeding well.

Examples of such prior art devices can be seen in U.S. Pat. Nos. 4,479,495, 5,295,996, 8,092,406.

In U.S. Pat. No. 4,479,495, an acupressure point stimulator can be seen having a flexible cinch strap with a fixed stimulator of a contoured configuration engaging against the user's body.

U.S. Pat. No. 5,295,996 discloses a pressure wrap device as a tourniquet with a spring-loaded projection on the strap against the pressure point.

U.S. Pat. No. 8,092,406 claims a therapeutic belt having various therapeutic components attached thereto for direct contact therapy to an area of need on the patient.

### SUMMARY OF THE INVENTION

An adjustable integrated hands-free compression device for applying compression on specific locations by conforming on the body with adaptable compression assembly having a band closure engagement track with one or more enabling bumper beads that are slidably disposed on the track to be maintained in applied orientation against the wound site during application. The bumper beads provide configured different outstanding profiles to apply pressure to the treatment site. A carrier patch for selective engagement on the body band provides adaptive insertable use of compression application for therapeutic inserts against the compression bumper beads engagement, as needed.

### DESCRIPTION OF THE DRAWINGS

FIG. 1 is a front, top, side perspective view of the adjustable wound compression device.

FIG. 2 is an enlarged partial top plan view thereof.

FIG. 3 is an exploded front perspective view of the adjustable compression device of the invention.

FIG. 4 is an enlarged perspective view of the compression bead track assembly.

FIG. 5 is a front elevational view of a body engagement band.

FIG. 6 is an enlarged side elevational view of a bumper bead.

FIG. 7 is an enlarged front perspective view of a therapeutic element carrier pouch.

FIG. 8 is a front elevational view of a therapeutic insert element.

### DETAILED DESCRIPTION OF THE INVENTION

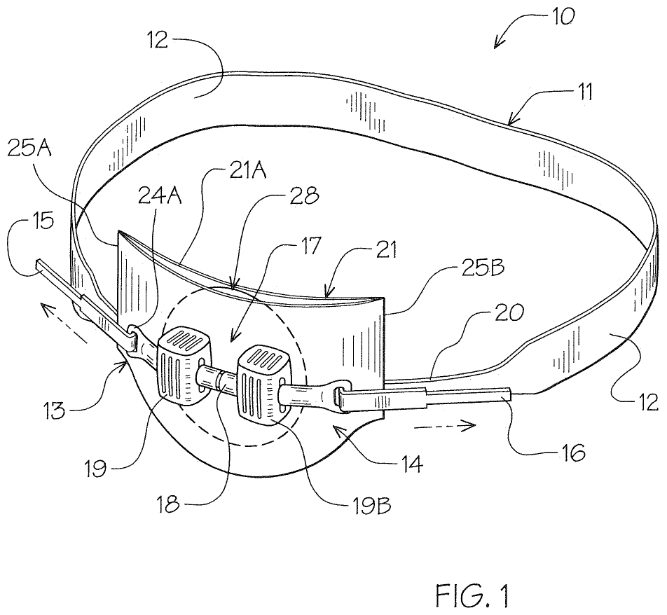

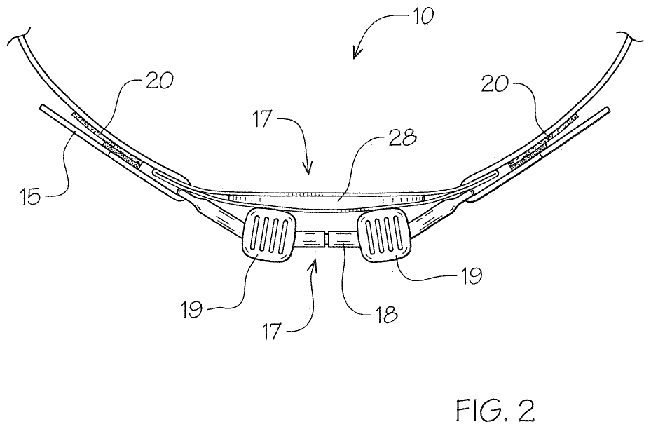

Referring to FIGS. 1 and 2 of the drawings, an adaptable compression device 10 of the invention can be seen capable of direct targeted compression on specific body wound locations as required inclusive of direct wound compression and therapeutic applications as will be described in detail hereinafter.

The compression device 10 of the invention has a body engagement band 11 which may be elastic having elongated body member 12 with oppositely disposed fastener engagement ends 13 and 14. Each of the fastener engagement ends 13 and 14 are of a reduced transverse dimension with user adjustment return connector tabs 15 and 16 respectively as best seen in FIGS. 1, 3 and 5 of the drawings.

A wound engagement compression assembly 17 has a compression assembly track 18 which is of a flexible profile corresponding to engagement openings through a pair of compression inducing bumper beads 19 which are slidably positioned thereon. The track 18 has, in this example, oppositely disposed end band registration connectors 20A and 20B with corresponding engagement apertures therein for receiving the hereinbefore described user adjustment return connector tabs 15 and 16 as will be described in greater detail hereinafter.

Accordingly, it will be seen that the band, return connector tabs 15 and 16 will have retainment elements such as hook and loop fabric fastener material 20 on opposing tab engagement surfaces commercially known as Velcro brand but not limited to example of tab retainment thereto.

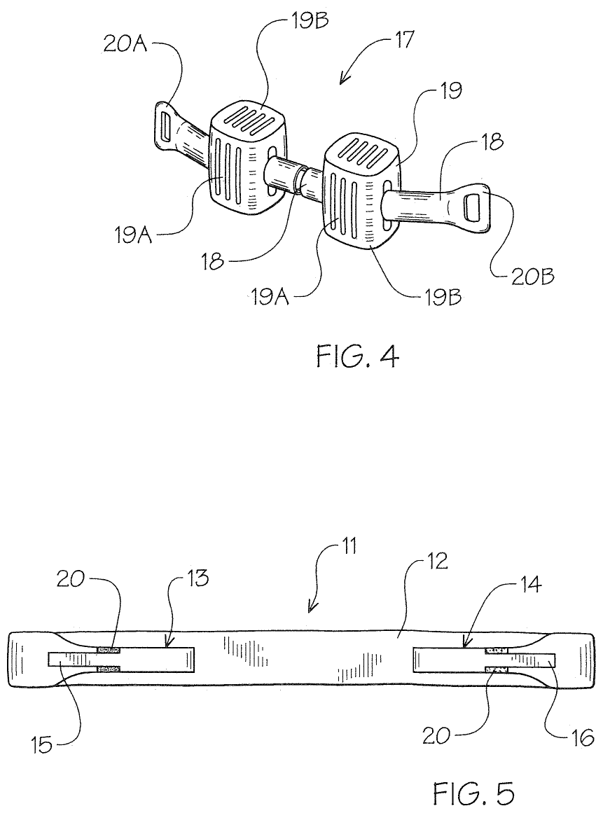

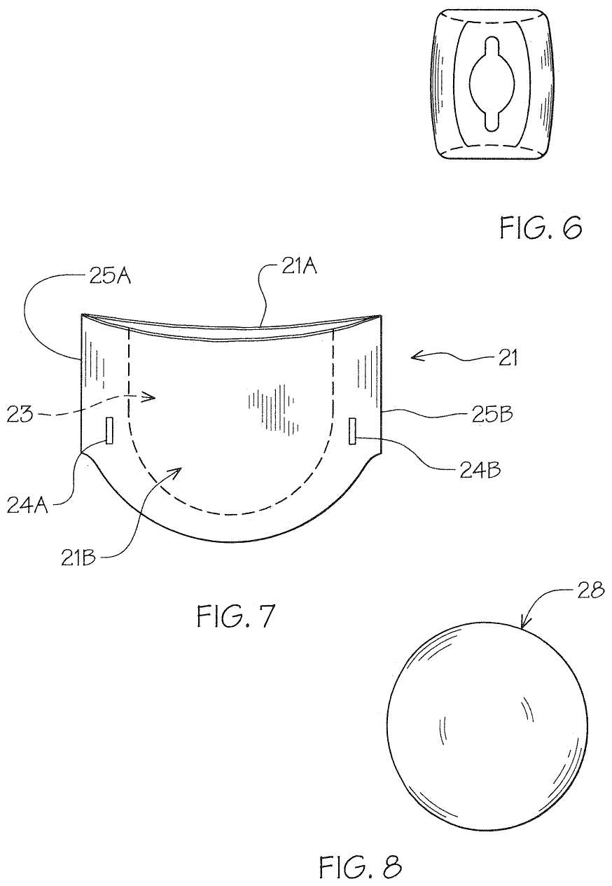

The track 18's profile, as noted, will be engaged through adaptive bumper bead 19 having, in this example, contoured surfaces with recessed finger grips 19A as well as protrusions such as ribs or nubs 19B on oppositely disposed sides, as best seen in FIGS. 4 and 6 of the drawings to aid in the bumper bead 19 positioning and retainment against associated compression treatment devices as to precise placement on the wound or trauma site.

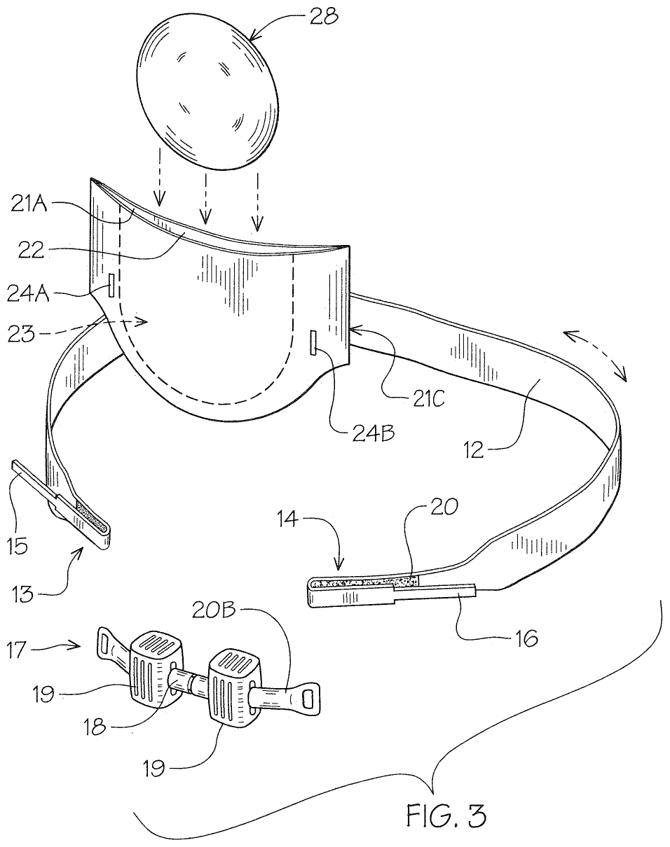

Referring now to FIGS. 3, 7 and 8 of the drawings, a carrier placement pouch 21 can be seen of a flexible material with a pouch receiving opening 21A along its upper edge 22 with front and back surfaces 21B and 21C defining a depending central pouch receiving area at 23. A pair of oppositely disposed support slots 24A and 24B are formed inwardly of the pouch respective side edges 25A and 25B in spaced relation to the defined center pouch receiving area 23 shown in broken lines in FIGS. 3 and 6 of the drawings.

The so configured “carrier” placement pouch 21 is engaged by the respective band return tabs 15 and 16 so as to be threadably received therethrough as seen in FIGS. 1 and 2 of the drawings positionally supporting the carrier placement pouch 21 between the tab ends of the band 11 and for direct engagement with the compression assembly 17 providing for a wide variety of therapeutic insert uses therewith. The carrier pouch 21 can be used to receive a variety of different therapeutic inserts such as, but not limited to a thermal pack 28, ice or heat, or other treatment modalities for the wound site as seen in FIGS. 1-3 of the drawings and FIG. 8.

In use, the compression device 10 utilizes the body or limb engagement band 11 and the compression assembly 17 in the following manner.

The adjustment end 13 of the band 11 with its adjustment return tab 15 is drawn through the back side of the carrier pouch slot 24A and through the aligned corresponding apertured track 18 registration end connector 20A with corresponding bumper beads 19 slidably positioned thereon and then returned for engagement with itself securing this portion of the compression assembly 17 thereto.

The band 11 is then placed around the area of the body, not shown, opposite the site of the incision or wound. The free engagement end 14 is correspondingly threaded through the remaining slot 24B of the carrier placement pouch 19 and correspondingly through the track 18 registration slot connectors 20B of the track 18. After confirming position on the wound or therapy site, the adjustment return tab 16 is correspondingly returned for engagement on itself by fastener material 20 thereby securing the compression assembly 17 in the desired position.

It will be seen that the hereinbefore described bumper beads 19's positional retainment adjustment is maintained by the bumper bead's contoured surfaces. Both of the band adjustment return tabs 15 and 16 may then be released and drawn away to the degree of compression assembly intensity desired and then effectively locked back into place against their respective fastening material 20 retainment surfaces as described. It will be evident that the compression assembly 17 does not require the inclusion of the carrier pouch 21, but may depending on the requirements of therapeutic inserts that may be used. The compression assembly 17, therefore, may be placed directly over the wound location on a provided appropriately shaped barrier, not shown, covering the wound or incision. The empty pouch 21 may also be employed to act as a barrier if so used or needed in such required wound use application venues that may occur.

It will be seen that the adaptable compression device 10 of the invention may be utilized in a wide manner of situations as well as adaptable size configurations to be adapted to alternate use applications. These would accommodate use in different compression placement where the health care provider may not easily reach and maintain the required compression needed.

Therefore I claim:

1. A therapeutic apparatus for compression application over a wound site comprising,
an elongated engagement band adjustably wrappable around a portion of a user's body having oppositely disposed free attachment ends,
a compressive assembly adjustably secured to said respective free ends of said band for engagement over said wound site,
at least one pressure inducement components slidably positioned on said compressive assembly.

2. The therapeutic apparatus for compression application over a wound site set forth in claim 1 wherein said compression assembly comprises,
an elongated track selectively secured between said respective free ends of said engagement band.

3. The therapeutic apparatus for compression application over a wound site set forth in claim 1 wherein said ends of said engagement band have respective elongated return tabs adjustably secured to themselves and said engagement band is elastic.

4. The therapeutic apparatus for compression application over a wound site set forth in claim 1 wherein said multiple pressure inducement components comprises,
contoured apertured beads adjustably positioned thereon.

5. The therapeutic apparatus for compression application over a wound site set forth in claim 1 wherein a therapeutic pouch in which therapeutic devices can be inserted is adjustably secured on said respective free ends of said bands for engagement by said compression assembly.

6. The therapeutic apparatus set forth in claim 5 wherein said therapeutic devices comprise, a thermal pack.

7. The therapeutic apparatus set forth in claim 5 wherein said therapeutic devices includes pressure creating component within said therapeutic pouch.

8. The therapeutic apparatus set forth in claim 3 wherein said return tabs adjustably secured to themselves by hook and loop fastener material.

9. The therapeutic apparatus set forth in claim 1 wherein said compression assembly is flexible.
